(19) United States
(12) Patent Application Publication
(10) Pub. No.: US 2022/0104856 A1
(43) Pub. Date: Apr. 7, 2022

(54) SCREW BONE IMPLANT

(71) Applicant: Anjali Investments LLC, Lutherville-Timonium, MD (US)

(72) Inventor: Amit R. Patel, Lutherville-Timonium, MD (US)

(21) Appl. No.: 17/421,104

(22) PCT Filed: Jan. 7, 2020
(86) PCT No.: PCT/US20/12602

§ 371 Date: Jul. 7, 2021

Related U.S. Application Data
(60) Provisional application No. 62/789,483, filed on Jan. 7, 2019.

(51) Int. Cl. A61B 17/86; A61B 17/70

(52) U.S. Cl. CPC A61B 17/864; A61B 17/70; A61B 17/866; A61B 17/863; A61B 17/861

(57) ABSTRACT

A screw implant is provided for the distraction, fusion, or compression of two adjacent bone structures or two adjacent bone fragments. The implant is a fully threaded screw with a headless proximal end having a drive engagement feature and a blunt distal end for insertion into bone or related tissue. The implant has roughened and porous surfaces throughout and is fully coated with hydroxyapatite and/or tri-calcium phosphate to allow for bone in-growth. The implant may have uniform low pitch cortical threads, or variable pitch threads, with low pitch cortical threads on one end and larger pitch cancellous threads on the other end. The implant may be used for the distraction of spinal vertebrae. The implants may have a cannulation channel and fenestrations.

### CROSS REFERENCE TO RELATED APPLICATION

This patent application claims the benefit of U.S. Patent Application 62/789,483 filed Jan. 7, 2019.

### FIELD OF THE INVENTION

The invention pertains to fully threaded screws as surgical implants for the distraction, compression, and/or fusion of vertebrae and other bones, joints, or bone segments.

### BACKGROUND

Cervical surgery since the late 1950s and early 1960s has steadily transitioned from primarily posterior based to primarily anterior based. Posterior based surgery has the following major advantages: minimal critical structures in the surgical field, familiar anatomy, and access to multiple levels. However, posterior based surgery has the following major disadvantages: dissection or approach related post-operative pain and large or limited arthrodesis option (for example, lateral mass screws and surface area available for fusion). Anterior based cervical surgery addresses some of the deficits, particularly pain and infection rates. Thus, the trend in the United States has been a shift towards more anterior cervical surgery and towards less posterior cervical surgery.

However, certain unique risks still remain with anterior surgery: injury to esophagus, injury to trachea, dysphagia/dysphonia, injury to recurrent laryngeal nerves, carotid artery injury, internal jugular vein injury, vagus nerve injury, adjacent level disease, non-unions, implant failure, implant prominence, dural injury, spinal cord injury to name a few. Thus, there has been a reemergence of posterior based surgery, particularly minimally invasive posterior surgery, to address the issues that may arise with anterior surgery.

Common indications for posterior minimally invasive surgery (MIS) include anterior cervical non-unions, patients at a high risk for non-unions (for example, smokers), and/or isolated foraminal stenosis which may lead to radiculopathy. In theory, given that the facet surface area of the posterior spine is equal to or larger than the surface area of the disc space anteriorly, one could minimize the rate of cervical non-unions or even treat anterior non-unions via a posterior MIS. Also, if a stenosis is present at the foraminal segment, indirect decompression via a posterior MIS would elevate the facet joint and increase the foraminal height as has been shown in the literature rather than the alternative anterior approach. Given the minimal disruption of soft tissue with posterior MIS, the infection rates and post-operative pain issues should match that of anterior based surgery.

Several wedge-type implants have been developed for treatment of degenerative disc disease (DDD) of the cervical spine (C3-C7), including cervical pseudarthrosis, such as “CAVUX®” (referred to “DTRAX®” in some literature), “HONOUR® ORB,” “Valeo® II C”, “UNIFLEX® Cervical cage” and others. These devices all require some degree of malleting for insertion. Some of these devices can be used for either anterior or posterior approaches (Smith, et al. “Anterior Cervical Pseudarthrosis Treated with Bilateral Posterior Cervical Cages,” Oper Neurosurg (Hagerstown). 2018 Mar. 1; 14(3):236-242. doi: 10.1093/ons/opx103, PMID: 28637309)

An alternative fusion technique involves the use of lateral mass screws in a posterior approach. For example, this includes DePuy/Synthes SYNAPSE™ and the Stryker OASYS® system. The major issue with any of these implants, despite the length of the screws on average being 10-16 mm, is related to the approach or exposure: an extremely wide dissection is required which may increase post-operative pain/disability and may increase the risk of infection. Furthermore, the screws themselves are primarily anchored in softer cancellous bone.

Related devices are also used for other bones besides the spine. See e.g., US 2018/0206897 A1, disclosing compression screws for bones not in the spine. Other prior art bone screws for distraction, compression, or fusion of bones or bone fragments are described in US 2009/0043308 and WO 2017/123753 A1.

### SUMMARY OF THE INVENTION

This disclosure addresses the shortcomings in the prior by providing a fully threaded screw implant intended for distraction or compression and fusion of the facet joints particularly for, but not limited to, the C3-C7 vertebrae, for the treatment of degenerative disc disease (DDD) of the cervical spine, stenosis, or pseudarthrosis. In addition, other bony fusions can be accomplished with the inventive implant.

In an embodiment, an implant is provided for the fusion of two adjacent bone structures or two adjacent bone fragments, comprising a fully threaded screw with a proximal end having a drive engagement feature and a distal end for insertion into bone or related tissue. In an embodiment, the screw has roughened surfaces throughout, and is fully coated with hydroxyapatite and/or tri-calcium phosphate, and is porous to allow for bone in-growth. In an embodiment, the proximal end of the screw is headless and the distal end to the head is flattened, rounded, or blunted. In an embodiment, the screw has uniform low pitch cortical threads, with with a major diameter of 3 mm to 7 mm, and an overall length of 5 mm to 65 mm; and the screw is made from titanium alloy or tantalum alloy with a similar modulus of elasticity to bone.

In an alternative embodiment, an implant for two adjacent bone structures or two adjacent bone fragments is provided, comprising a fully threaded screw with a proximal end having a drive engagement feature and a distal end for insertion into bone or related tissue. In an embodiment, the screw has roughened surfaces throughout, is fully coated with hydroxyapatite and/or tri-calcium phosphate and is porous to allow for bone in-growth. In an embodiment, the proximal end of the screw is headless and the distal end to the head is flattened, rounded, or blunted. In an embodiment, the screw has variable pitch threads, having low pitch cortical threads distal to the head, and larger pitch cancellous threads proximal to the head, In an embodiment, the screw has a uniform major diameter of 3 mm to 7 mm for the entire length of the screw, and an overall length of 5 mm to 65 mm. In an embodiment, wherein the screw is made from titanium alloy or tantalum alloy with a similar modulus of elasticity to bone.

In an alternative embodiment, an implant for fusion of two adjacent bone structures or two adjacent bone fragments is provided, comprising a fully threaded screw with a proximal end having a drive engagement feature and a distal end for insertion into bone or related tissue. In an embodiment, the screw has roughened surfaces throughout, is fully coated with hydroxyapatite and/or tri-calcium phosphate and is porous to allow for bone in-growth. In an embodiment, the proximal end of the screw is headless and the distal end to the head is flattened, rounded, or blunted. In an embodiment, the screw has variable pitch threads, having large pitch cancellous threads distal to the head, and low pitch cortical threads proximal to the head, In an embodiment, the screw has a uniform major diameter of 3 mm to 7 mm for the entire length, and an overall length of 5 mm to 65 mm. In an embodiment, the screw is made from titanium alloy or tantalum alloy with a similar modulus of elasticity to bone.

In an embodiment, the screw implants have a major diameter (major diameter) of 4 mm to 5 mm. In an embodiment, the screw implants have an overall length is selected from 10 mm, 12 mm, 15 mm, or any other length from 15 mm to 65 mm. In an embodiment, the screw implants are solid without a cannulation channel. In an embodiment, the screw implants are cannulated with a channel through the center to allow placement with a guidewire. In an embodiment, the screw implants have one or more fenestrations, with or without cannulation. The fenestrations may be slots or perforations to allow for bone grafting and bony through-growth.

In an embodiment, the implant is fully threaded screw-like implant with low pitch cortical threads with major diameter of 3 mm to 7 mm and a length of 5 mm to 65 mm. In an embodiment, the implant would have low-pitch threads (close threads) and low crests (to minimize difference between inner and outer diameters) similar to commonly seen set screws. In an alternative embodiment, the implant could have variable pitch threads to allow for distraction of the facet joints if placed across a joint line (low pitch threads distally and large pitch threads proximally). In another embodiment, the implant could have variable pitch threads to allow for compression of the facet joints if placed across a joint line (large pitch threads distally and low pitch threads proximally).

In an embodiment, the implant would have a leading end (distal) that is blunt and a trailing (proximal) end that allows for a driver to engage the implant, for example a hexagonal screwdriver.

In an embodiment, the implant is fully porous with pore sizes ranging in 100 to 900 μm to facilitate in-growth and have a porosity to mimic host cancellous bone.

In an embodiment, the implant is made of titanium or tantalum or an alloy thereof to match the modulus of elasticity of bone.

In an embodiment, the implant would be coated with HA (hydroxyapatite) and/or TCP (tri-calcium phosphate) to facilitate osteo-conduction.

In an embodiment, the implant is threaded to allow for simpler in-line insertion in between the facets and into the joint space. Alternatively, the implant insertion can be perpendicular to the joint line in the embodiment with variable threads to allow for joint distraction or compression.

In an embodiment, the implant may be cannulated to allow placement with a guidewire.

In an embodiment, the implant may have fenestrations or slots or perforations to allow for bone grafting and bony through-growth.

### DESCRIPTION OF THE DRAWINGS

FIG. 1 is an elevation view of the inventive implant with uniform cortical threads

FIG. 2. is an elevation view of the inventive implant with variable pitch threads, with cancellous threads at the proximal end, and cortical threads at the distal end.

FIG. 3 elevation view of the inventive implant with variable pitch threads, with cortical threads at the proximal end, and cancellous threads at the distal end.

FIG. 4 is a perspective of the proximal end.

FIG. 5 is a perspective of the distal end.

FIG. 6 is an elevation view of a screw according to this invention having fenestrations.

FIG. 7 is elevation view of a screw according to this invention having alternative fenestrations.

FIG. 8 shows an inline implant configuration between two vertebrae.

FIG. 9 shows a perpendicular implant configuration between two vertebrae.

### DETAILED DESCRIPTION

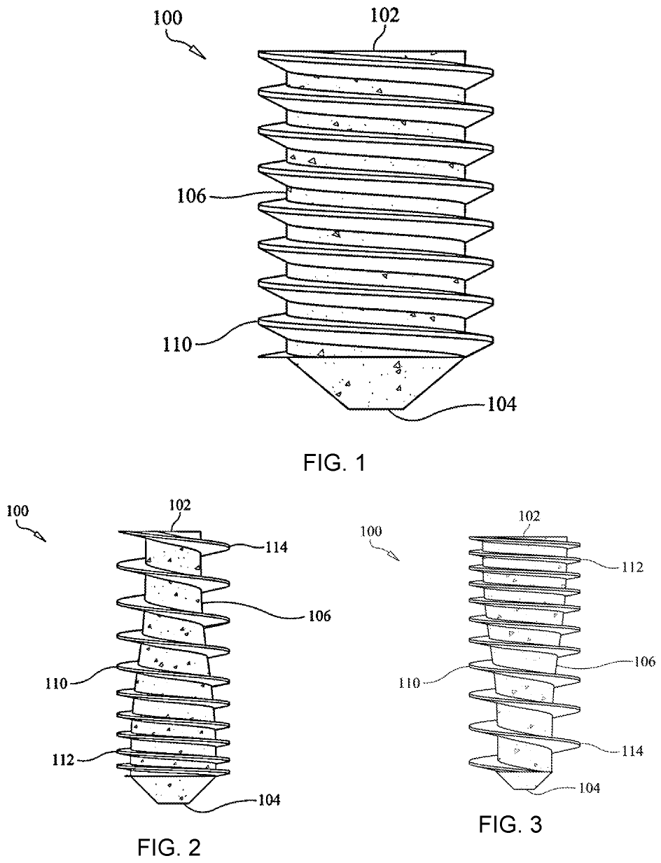

This invention provides an implant for fusion of cervical spinal vertebrae, wherein the implant is a fully threaded screw with a cylindrical body as shown in FIGS. 1-7, having a uniform major diameter. For orientation, the screw has a distal first end that is flattened, rounded, or blunted for insertion into bone tissue. The screw has a proximal second end that is headless and has a drive engagement feature, such as a hexagonal head, star head, or Phillips head. The designations of distal and proximal are in relation to the surgeon.

The inventive implants may be used for distraction, fusion, and bone compression of adjacent bones or bone fragments, including vertebrae. In an embodiment, the screws are inserted into the posterior facet joint between two vertebrae. Alternatively, the screws can be inserted across a facet joint using a drilled hole and optionally a guide wire. In contrast to the prior art devices discussed above, such as the CAVUX®, HONOUR® ORB, Valeo® II C, UNIFLEX® Cervical cage, which require malleting or tamping during insertion, the inventive implants 100 are inserted by being screwed into position. All of the above referenced prior art devices are cages and have a boxy or wedge shape to some extent. All of the cage-like devices must be malleted into position. The inventive devices which are screwed into position for vertebral distraction afford a much greater degree of control for the surgeon. Malleting also has the disadvantage that fracturing or chipping of bone at the facet joint is a common adverse event. If this occurs, invasive remediation is required such as open or traditional fusion with screws and rods.

In another embodiment, the implants such as those illustrated in FIG. 3 can be used for compression of two adjacent bone structures or two adjacent bone fragments, not limited to the spine. The embodiment of FIG. 3 has large pitch cancellous threads distal to the head and small pitch cortical threads proximal to the head.

The inventive device has significant advantages over the screws disclosed in US 2009/0312763 A1 (763) at FIGS. 39-47. The '763 devices do not have the osteoconductive hydroxyapatite or TCP coatings of the inventive implants and do not have porous surfaces and roughened surfaces.

The inventive devices have features to facilitate osseoincorporation or osseointegration of the implant into bone tissue. In an embodiment, the inventive implants are equipped with a roughened surface with a HA and/or TCP to facilitate bone ongrowth on the implant. (Jung Taek Kim, MD and Jeong Joon Yoo, “Implant Design in Cementless Hip Arthroplasty,” Hip Pelvis. 2016 June; 28(2): 65-75, doi: 10.5371/hp.2016.28.2.65 (see p. 65)) The porous surfaces facilitate bone ingrowth. Bone ingrowth refers to the formation of bone within an irregular surface of an implant, which improves the implant's integration into bone. The presence of a porous-coated implant evokes a cellular and physiological response that resembles the healing cascade of cancellous defects. In porous implants, the void spaces are filled with newly formed bone tissue when the implants are stable. Fenestrations promote bone through-growth, meaning bone growth through the fenestrations.

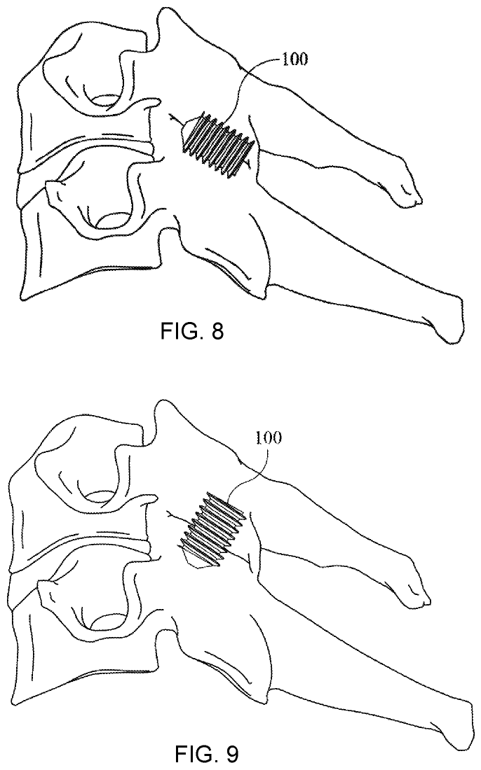

The insertion of the implants 100 may take two or more configurations. One embodiment is shown in FIG. 8 with the implant inserted inline into a facet joint. Another embodiment is shown in FIG. 9 with the implant inserted into a drilled bore across a facet joint, for example in a generally perpendicular orientation to the facet joint. In either embodiment, two or more insertions may be performed, so that for example, one or two implants may be inserted into or perpendicular to a facet joint on each side of the vertebra.

With either installation configuration, the width of the dissection can be narrower than is required with the DePuy/Synthes SYNAPSE™ and the Stryker OASYS® system.

FIGS. 1-7 show the inventive implant 100 with the proximal end 102 and distal end 104, and threads 110 on body 106. In an embodiment, the inventive implant 100 is a fully threaded screw.

In the embodiment shown in FIG. 1, the threads on implant 100 are uniform cortical threads 112. In an embodiment, cortical threads 112 may have a pitch of 1.25 mm to 1.75 mm, a thread height of 0.5 mm to 1.5 mm. The major diameter of the implant may be 3 mm to 7 mm. The overall length of the implant may be 5 mm to 65 mm. In an embodiment, the screw is 8, 10, 12 or 15 mm long. In embodiment, the implant may be any other length between 15 mm and 65 mm, such as 20 mm, 25 mm, 30 mm, 35 mm, 40 mm, 45 mm, 50 mm, 55 mm, 60 mm or 65 mm. In an embodiment, the screw is 4-5 mm wide. In an embodiment, the screw has a 3.1 mm minor diameter and a 4.5 mm major diameter.

The “major diameter” (Dₘₐⱼ) is defined as the maximum outside diameter of the threads. The Dₘₐⱼ, is the larger of two extreme diameters delimiting the height of the thread profile, as a cross-sectional view is taken in a plane containing the axis of the threads. The “minor diameter” (Dₘᵢₙ) is the diameter of the core of the implant, i.e., at the bottom of the threads. The Dₘᵢₙ is the lower extreme diameter of the thread. As used herein, the “core diameter” or “shaft diameter” is equivalent to the minor diameter. In an embodiment, the implant has a uniform major diameter for the entire length, except for the rounded, flattened, or blunted distal tip. “Thread height” is defined at Dₘₐⱼ-Dₘᵢₙ. In an embodiment, the Dₘᵢₙ may vary, particularly in embodiments with variable thread pitches, in order to keep the Dₘₐⱼ uniform. In such embodiments, the Dₘᵢₙ will be smaller in sections of the screw with larger cancellous threads than in sections of the screw with smaller cortical threads.

In an embodiment, the threads may have low crests, meaning shallow threads, that is, meaning the difference between the major and minor diameter will be smaller than a normal 1.25 mm to 1.75 mm pitch thread. This is similar to commonly used orthopedic set screws or cortex screws.

In an embodiment, the distal end 104 of implant 100 is flattened, rounded, or blunted. This is desirable to minimize damage during insertion and reduce the tolerance necessary to insert the implant without unnecessary damage at the implant site.

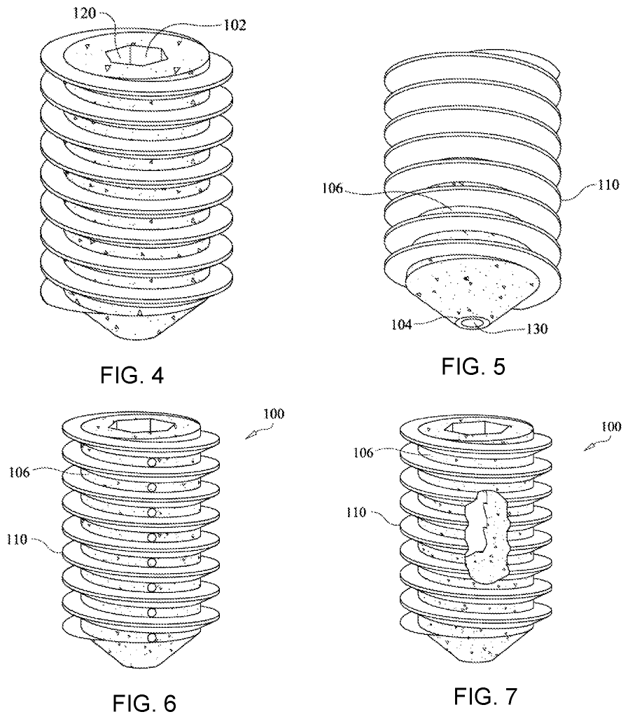

In an embodiment the proximal end 102 of the implant is headless and includes a drive engagement feature 120, such as a hexagonal head, star head, or Phillips head, for the insertion of a screwdriver to rotationally drive the implant into position. However, other shaped features are also contemplated (both male and female). For example, the drive engagement feature 120 can include a cruciate shape, square shape, six-point star shape, or the like. Where a hexagonal (or “hex”) feature is used (e.g., 120 in FIG. 4), the headless compression screw 100 can be driven by a driver that can include a male hexagonal engagement portion. The driver can engage the headless compression screw, such as via the female hex feature, and can be capable of driving the headless compression screw across a bone fracture.

In an embodiment, the implant 100 has no external head (headless), meaning the drive engagement feature 120 is recessed into the body of the implant and there is no portion of the device outside the shape of the cylinder or cone 106 (i.e., the minor diameter) needed to accommodate the drive engagement feature. This is important because in an embodiment, the inventive implant 100 may be driven entirely into the bone, with no portion of the screw exterior to the natural surface of the bone (FIGS. 8-9).

FIG. 2 shows an embodiment of the inventive implant with variable pitch threads such that the thread pitch is smaller at the distal end, and larger at the proximal end. In an embodiment, the major diameter is uniform the entire length of the device, which implies that the minor diameter 106 narrows towards the proximal end to accommodate the larger thread height of larger pitch threads at the proximal end. This is the opposite configuration to certain compression screws such as the “Arthrex® Compression FT” screws, indicated for repairing intra-articular and extra-articular fractures and nonunions of small bones and small bone fragments, arthrodesis and osteotomies, in hands, feet, and extremities (US2009/0043308 A1). In contrast to the Arthrex screws, the inventive screws are for a different purpose, distraction and spinal fusion, in particular for fusion of vertebrae such as across a facet joint between two vertebrae.

In the embodiment shown in FIG. 2, the threads on implant 100 on the distal end are low-pitch cortical threads 112, and the threads on the proximal end are larger pitch cancellous threads 114. In an embodiment, the cortical threads 112 have a pitch of 1.25 mm to 1.75 mm, a thread height of 0.5 mm to 1.5 mm, and the cancellous threads 114 have a pitch of 2.0 mm to 4.0 mm and a thread height of 1.5 mm to 3.0 mm. In an embodiment, the device in FIG. 2 has a uniform major diameter of 3 mm to 7 mm for the entire length except the tip, and a length of 5 mm to 65 mm.

In an embodiment, the implant of FIG. 2 has a major diameter of 4-5 mm and a length of 8-12 mm. This size is particularly desirable for use to fuse the C3-C7 vertebra by placement in a drilled hole across joint. An embodiment of this placement is shown in FIG. 9. The implant placement may be approximately perpendicular to the plane of the joint as shown in FIG. 9, or at an angle to the plane of the joint. For other locations, the implant may be longer, and be made to any length up to about 65 mm, such as 20 mm, 30 mm, 40 mm, 50 mm, 60 mm.

In an embodiment as shown in FIG. 3, the threads on implant 100 on the distal end are larger pitch cancellous threads 114, and the threads on the proximal end are low-pitch cortical threads 112. In an embodiment, the cancellous threads have a pitch of 2.0 mm to 4.0 mm and a thread height of 1.5 mm to 3.0 mm and the cortical threads have a pitch of 1.25 mm to 1.75 mm, a thread height of 0.5 mm to 1.5 mm. In an embodiment, the implant of FIG. 3 has a uniform major diameter of 3 mm to 7 mm for the entire length except the distal tip, and a length of 5 mm to 65 mm. In an embodiment, the device of FIG. 3 has uniform major diameter of about 4-5 mm. In an embodiment, the device of FIG. 3 may be made to any length up to about 65 mm, such as 20 mm, 30 mm, 40 mm, 50 mm, 60 mm.

In an embodiment, the device of FIG. 3 is a compression screw, that draws two bones or bone fragments together and compresses the joint for fusion. As the screw is inserted and rotated, the cancellous threads pull the distal fragment towards the proximal fragment.

In an embodiment, the device of FIGS. 1-7 may be solid, or may be cannulated, that is, with a channel 130 (FIG. 5) running the length of the screw, for the insertion of a guidewire. During insertion, the implant may be placed into position with a guidewire. A common surgical technique with bone implants is for the surgeon to drill a small diameter bore into the desired site of the implant and then place a guidewire through the drilled trajectory. Another technique would be to insert the guidewire and then drill over the guidewire with a cannulated drill bit. Under fluoroscopic guidance, the placement of this bore or guidewire can be monitored and assessed for an appropriate trajectory. The guide wire may also be used to ensure proper final placement of the implant. An advantage to this technique is that it involves drilling a pilot hole, so if the hole is not at the exact correct spot, another pilot hole can be drilled. An additional advantage is that the placement of the implant can be viewed fluoroscopically to ensure the correct placement, prior to drilling the main borehole.

The inventive implant may be equipped with features to enhance bone in-growth. In an embodiment, the implant may be fabricated from a porous material known to enhance bone in-growth. In an embodiment, the implant may have a uniformly roughened surface to enhance bone on-growth and provide a scratch fit or interference fit. The implant may also be coated with an osteoconductive coating and equipped with fenestrations.

In an embodiment, the device of FIGS. 1-7 may include one or more fenestrations that may be slots or perforations to allow for bone grafting and bone through-growth. Various embodiments are shown in FIGS. 6-7. FIG. 6 shows a series of small holes that could be drilled into the device. FIG. 7 shows a large slot penetrating through the entire device. The slot in FIG. 7 is illustrated with an uneven surface. The slots or perforations may penetrate through the entire width of the device, or only part way through.

In an embodiment, the entire implant may be fabricated from a medically compatible tantalum, titanium, tantalum alloy, or titanium alloy. For example, an appropriate titanium alloy may be titanium 6AL4V and 6AL4V ELI (ASTM Standard F1472, https://www.astm.org/Standards/F1472.htm (see also https://en.wikipedia.org/wiki/Ti-6Al-4V)), which are alloys made with about 6% aluminum and 4% vanadium. An appropriate tantalum alloy may be tantalum alloyed with 2.5% to 10% tungsten, or 40% niobium. These materials are known to have good biocompatibility and match the modulus of elasticity of bone. In an embodiment, the implant may be manufactured from a titanium alloy in accordance with ASTM F136, or where exterior surfaces are coated with medical-grade commercially pure titanium (CP Ti) per ASTM F1580.

In an embodiment, titanium or tantalum alloys can be made with roughened and porous surfaces. See e.g., https://www.slideshare.net/sameerashar9/uncemented-femoral-stem and Vasconcellos L M et al. “Evaluation of bone ingrowth into porous titanium implant: histomorphometric analysis in rabbits,” Braz Oral Res. 2010 October-December; 24(4):399-405, DOI: 10.1590/s1806-83242010000400005.

In an embodiment, all surfaces of the implant may be roughened with a macro surface roughness. This may be accomplished with a technique such as grit blasting, acid etching, or plasma spray coating (also called thermal spray coating). The rough surfaces are indicated in the drawings by the random dot pattern shown throughout.

In an embodiment, all surfaces of the implant 100 are coated with hydroxyapatite (HA) and/or tricalcium phosphate (TCP). HA and TCP are well known as osteoconductive materials that encourage bone growth.

In an embodiment, the implant may be fabricated from a porous material known to enhance bone in-growth, for example with pore sizes ranging in 100 to 900 μm to facilitate in-growth and have a porosity of 60-65% to mimic cancellous bone. Porosity may be created by mechanical manipulation of the screws, such with micro-drilling or laser drilling in an uneven pattern. The porosity is shown in the figures by the larger surface imperfections depicted as triangles.

The combination of surface roughness, HA or TCP coating, and porosity will facilitate bone in-growth which is desirable for fusion.

1. An implant for fusion of two adjacent bone structures or two adjacent bone fragments, comprising a fully threaded screw with a proximal end having a drive engagement feature and a distal end for insertion into bone or related tissue, wherein
a. the screw has roughened surfaces throughout, is fully coated with hydroxyapatite and/or tri-calcium phosphate, and is porous to allow for bone in-growth;
b. the proximal end of the screw is headless and the distal end to the head is flattened, rounded, or blunted;
c. the screw has uniform low pitch cortical threads, with a major diameter of 3 mm to 7 mm, and a length of 5 mm to 65 mm; and
d. the screw is made from titanium alloy or tantalum alloy with a similar modulus of elasticity to bone.

2. An implant for fusion of two adjacent bone structures or two adjacent bone fragments, comprising a fully threaded screw with a proximal end having a drive engagement feature and a distal end for insertion into bone or related tissue; wherein
a. the screw has roughened surfaces throughout, is fully coated with hydroxyapatite and/or tri-calcium phosphate, and is porous to allow for bone in-growth;
b. the proximal end of the screw is headless and the distal end to the head is flattened, rounded, or blunted;
c. the screw has variable pitch threads, having low pitch cortical threads distal to the head, and larger pitch cancellous threads proximal to the head, wherein; the screw has a uniform major diameter of 3 mm to 7 mm for the entire length, and a length of 5 mm to 65 mm;
d. wherein the screw is made from titanium alloy or tantalum alloy with a similar modulus of elasticity to bone.

3. An implant for fusion of two adjacent bone structures or two adjacent bone fragments, comprising a fully threaded screw with a proximal end having a drive engagement feature and a distal end for insertion into bone or related tissue; wherein
a. the screw has roughened surfaces throughout, is fully coated with hydroxyapatite and/or tri-calcium phosphate, and is porous to allow for bone in-growth;
b. the proximal end of the screw is headless and the distal end to the head is flattened, rounded, or blunted;
c. the screw has variable pitch threads, having large pitch cancellous threads distal to the head, and low pitch cortical threads proximal to the head, wherein; the screw has a uniform major diameter of 3 mm to 7 mm for the entire length, and a length of 5 mm to 65 mm;
d. wherein the screw is made from titanium alloy or tantalum alloy with a similar modulus of elasticity to bone.

4. The screw of claims 1-3, wherein the screw diameter (major diameter) is 4 mm to 5 mm.

5. The screw of claims 1-3, wherein the screw length is selected from 10 mm, 12 mm, and 15 mm.

6. The screw of claims 1-3, wherein the implant is solid without cannulation.

7. The screw of claims 1-3, wherein the implant is cannulating to allow placement with a guidewire.

8. The screw of claims 1-3, wherein the screw comprises one or more fenestrations with or without cannulation.

9. A method of fusing spinal vertebrae, comprising the implant of claim 1 inserted in-line with a joint between two vertebrae.

10. A method of fusing cervical spinal vertebrae, comprising the implant of claim 1 inserted in-line with a facet joint between two vertebrae

11. A method of fusing cervical spinal vertebrae, comprising the implant of claim 1 inserted in-line with a facet joint in the C3-C7 vertebrae.

12. A method of fusing spinal vertebrae comprising the implant of claim 2 or claim 3 inserted across a joint between two vertebrae.

13. A method of fusing cervical spinal vertebrae comprising the implant of claim 2 or claim 3 inserted across a facet joint between two vertebrae.

14. A method of fusing cervical spinal vertebrae comprising the implant of claim 2 or claim 3 inserted across a facet joint in the C3-C7 vertebrae.

15. A method of fusing two bones or two bone fragments comprising the implant of claim 2 or claim 3 inserted across a joint between the two bones or bone fragments.

16. A method of fusing two disjointed bones or bone fragments comprising inserting the implant of claim 2 or 3 across a joint between the bone fragments wherein the implant stabilizes or compresses the joint.
